SEQUENCE LISTING (4 sequences)

SEQ ID NO: 1 - DNA, 20 bp, synthetic construct
  misc_feature 1..20: note = beta-actin forward primer
  source 1..20: mol_type = other DNA
ccaccatgtacccaggcatt

SEQ ID NO: 2 - DNA, 20 bp, synthetic construct
  misc_feature 1..20: note = beta-actin reverse primer
  source 1..20: mol_type = other DNA
cggactcatcgtactcctgc

SEQ ID NO: 3 - DNA, 19 bp, synthetic construct
  misc_feature 1..19: note = RAGE forward primer
  source 1..19: mol_type = other DNA
aggtggggacatgtgtgtc

SEQ ID NO: 4 - DNA, 20 bp, synthetic construct
  misc_feature 1..20: note = RAGE reverse primer
  source 1..20: mol_type = other DNA
tctcagggtgtctcctggtc

(19) United States
(12) Patent Application Publication
(10) Pub. No.: US 2023/0190677 A1
(43) Pub. Date: Jun. 22, 2023

(54) PHARMACEUTICAL COMPOSITION FOR PREVENTING OR TREATING AGING-RELATED DISEASES

(71) Applicant: INDUSTRY ACADEMIC COOPERATION FOUNDATION CHOSUN UNIVERSITY, Gwangju (KR)

(72) Inventors: Gwang Won CHO, Gwangju (KR); Nagarajan MAHARAJAN, Gwangju (KR)

(21) Appl. No.: 17/858,233

(22) Filed: Jul. 6, 2022

(30) Foreign Application Priority Data
Jul. 6, 2021 (KR) 10-2021-0088555

(51) Int. Cl. A61K 31/12; A61P 39/06; A23L 33/105; A61K 8/35; A61Q 19/08

(52) U.S. Cl. CPC A61K 31/12; A61P 39/06; A23L 33/105; A61K 8/35; A61Q 19/08; A61K 2800/522

(57) ABSTRACT

A method for treatment of aging-related disease includes administering a composition including a compound represented by Formula 1 or a pharmaceutically, sitologically or cosmetically acceptable salt thereof to a subject in need thereof. The composition has excellent AMP-activated protein kinase (AMPK) and autophagy activation effects, thereby being helpful to prevent aging.

### CROSS REFERENCE TO RELATED APPLICATIONS AND CLAIM OF PRIORITY

This application claims the benefit under 35 USC § 119(a) of Korean patent Application No. 10-2021-0088555, filed on Jul. 6, 2021, in the Korean Intellectual Property Office, the entire disclosure of which is incorporated herein by reference for all purposes.

### BACKGROUND

### 1. Field of the Invention

The present invention relates to a pharmaceutical composition for prevention or treatment of aging-related diseases.

### 2. Description of the Related Art

Due to the continuation of the global aging phenomenon, it is predicted that people aged 65 or more will enter an aged society that accounts for more than 14% of the total population since 2018. The increase in the elderly population means that the percentage of the population suffering from chronic diseases or with reduced biomechanical function is increasing. Therefore, the development of aging industry such as medicines, functional foods, cosmetics, etc. in order to improve health as well as desires for the quality of life is expected.

Further, for median-aged population, there is a growing demand to improve their various bio-indicators relevant to aging before they get older and to live in a healthier and younger state. Moreover, since the proportion of women is increasing in an aspect of gender distribution with economic and social activities, the development of anti-aging materials and products for women is very great in demand. Among them, a demand for products with definite verification of scientific efficacy is now being increased.

In order to meet the demand for anti-aging, it is necessary to identify major characteristics of aged tissues and cells and to find a way to improve the same. A major feature represented in the aged tissues or cells to constitute organs is a drastic decrease in autophagy activity. As autophagy activity in the cells is decreased, cell viability and activity are also rapidly lowered thus to exhibit a morphological pattern of aging, which in turn may be developed into cell aging, neurodegenerative diseases or metabolic diseases such as type 2 diabetes.

Autophagy refers to a mechanism that regenerates energy and removes damaged substances by decomposing the aged or damaged cellular materials and organs when an intracellular energy source is depleted or intracellular stress factors are excessively generated, while enabling normal cells to be maintained. Recently, various researches have reported that, as the aging progresses or the aging accelerates, the intracellular autophagy activity is rapidly decreased. On the other hand, when suppressing the autophagy, aged mitochondria and/or misfolded proteins accumulate excessively in the cells, and thus free radicals and oxidative stress are increased, hence resulting in increased cell death (apoptosis) and acceleration of aging.

### SUMMARY

An object of the present invention is to provide a pharmaceutical composition for prevention or treatment of aging-related diseases.

In addition, another object of the present invention is to provide an anti-aging food composition.

Further, another object of the present invention is to provide an anti-aging cosmetic composition.

Furthermore, another object of the present invention is to provide a method for treatment of aging-related diseases.

To achieve the above objects, the following technical solutions are adopted in the present invention.

1. A pharmaceutical composition for prevention or treatment of aging-related diseases including a compound represented by Formula 1 below or a pharmaceutically acceptable salt thereof:

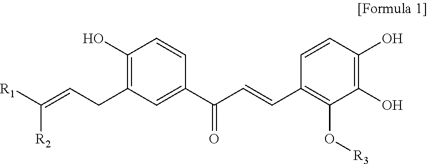

(wherein R₁ is H or C1 to C6 alkyl, R₂ is H or C1 to C6 alkyl, and R₃ is H or C1 to C6 alkyl).

2. The composition according to the above 1, wherein the aging-related disease is one selected from the group consisting of neurodegenerative diseases, cognitive functional disorder due to aging and skin-related diseases.

3. The composition according to the above 2, wherein the neurodegenerative disease is one selected from the group consisting of dementia, Alzheimer's disease, Huntington's disease and Parkinson's disease.

4. The composition according to the above 2, wherein a cognitive function in regard to the cognitive functional disorder is one selected from the group consisting of perception, memory, attention, speech comprehension, speech generation, reading comprehension, creation imagery, learning and reasoning.

5. An anti-aging food composition including a compound represented by Formula 1 below or a sitologically acceptable salt thereof:

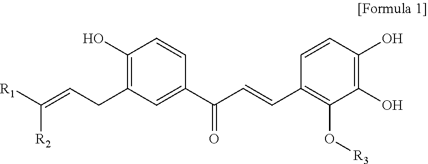

(wherein R₁ is H or C1 to C6 alkyl, R₂ is H or C1 to C6 alkyl, and R₃ is H or C1 to C6 alkyl).

6. An anti-aging cosmetic composition including a compound represented by Formula 1 below or a cosmetically acceptable salt thereof:

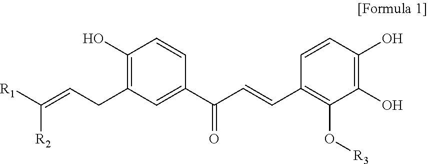

(wherein R₁ is H or C1 to C6 alkyl, R₂ is H or C1 to C6 alkyl, and R₃ is H or C1 to C6 alkyl).

7. A method for prevention or treatment of aging-related diseases including administering a compound represented by Formula 1 below or a pharmaceutically acceptable salt thereof to a subject in need thereof:

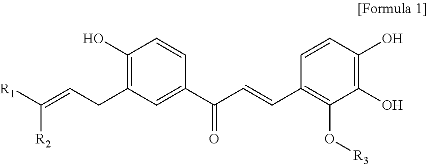

(wherein R₁ is H or C1 to C6 alkyl, R₂ is H or C1 to C6 alkyl, and R₃ is H or C1 to C6 alkyl).

The pharmaceutical composition, food composition and cosmetic composition of the present invention have autophagy-inducing effects through excellent activation of AMP-activated protein kinase (AMPK).

### BRIEF DESCRIPTION OF THE DRAWINGS

The above and other objects, features and other advantages of the present invention will be more clearly understood from the following detailed description taken in conjunction with the accompanying drawings, in which:

FIGS. 1A to 1E illustrate results of confirming protective effects of Licochalcone D to human bone marrow mesenchymal stem cells (hBM-MSCs) under H₂O₂-induced oxidative stress;

FIGS. 2A to 2C illustrate results of confirming anti-aging effects of Licochalcone D to hBM-MSCs through a decrease in expression of β-galactosidase;

FIGS. 3A to 3F illustrate results of confirming anti-aging effects of Licochalcone D to hBM-MSCs through decreases in expression of p16, p21 and p53 and activation of AMP-activated protein kinase (AMPK);

FIG. 4A to 4D illustrate results of confirming anti-aging effects of Licochalcone D to hBM-MSCs through extent of autophagy activation;

FIGS. 5A to 5C illustrate results of confirming improvement of learning and amnestic disorder by Licochalcone D in a mouse model with induced learning and amnestic disorder by D-galactose (D-Gal), through a decrease in expression of receptor for advanced glycation endproducts (RAGE);

FIGS. 6A to 6C illustrate results of confirming improvement effects of behavior disorder in mice by Licochalcone D in a mouse model with induced learning and amnestic disorder by D-galactose (D-Gal), through a Morris water maze (MWM) test;

FIGS. 7A to 7E illustrate results of confirming anti-aging effects of Licochalcone D to mouse heart tissues with aging induced by D-Gal through decreases in expression of p21 and p53, respectively (FIGS. 7A to 7C) and activation of AMPK (FIGS. 7D and 7E), while FIGS. 7F to 7J illustrate results of confirming anti-aging effects of Licochalcone D to mouse hippocampal tissues with aging induced by D-Gal through decreases in expression of p21 and p53, respectively (FIGS. 7F to 71) and activation of AMPK (FIGS. 71 and 7J);

FIGS. 8A to 8D illustrate results of confirming anti-aging effects of Licochalcone D to mouse heart tissues with aging induced by D-Gal through autophagy activation; and

FIG. 9 is a schematic diagram showing aging improvement effects of Licochalcone D to stem cells and aged mice.

### DETAILED DESCRIPTION

Hereinafter, the present invention will be described in detail.

The present invention relates to a pharmaceutical composition for prevention or treatment of aging-related diseases.

The pharmaceutical composition may include a compound represented by Formula 1 below or a pharmaceutically acceptable salt thereof:

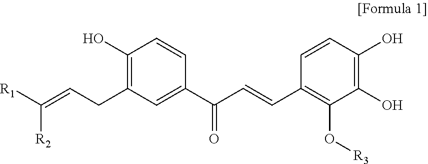

(wherein R₁ is H or C1 to C6 alkyl, R₂ is H or C1 to C6 alkyl, and R₃ is H or C1 to C6 alkyl).

In Formula 1 above, R₁ may be H or C1 to C6 alkyl. In Formula 1 above, R₁ may be H or C1 to C3 alkyl.

In Formula 1 above, R₂ may be H or C1 to C6 alkyl. In Formula 1 above, R₂ may be H or C1 to C3 alkyl.

In Formula 1 above, R₃ may be H or C1 to C6 alkyl. In Formula 1 above, R₃ may be H or C1 to C3 alkyl.

In Formula 1 above, R₁ may be H or C1 to C3 alkyl, R₂ may be H or C1 to C3 alkyl, and R₃ may be H or C1 to C3 alkyl.

The compound represented by Formula 1 may be a material extracted from natural substances, or may be chemically synthesized.

According to one embodiment, in Formula 1 above, R₁ may be C1 alkyl, R₂ may be C1 alkyl and R₃ may be C1 alkyl. In Formula 1, the structure wherein R₁ is C1 alkyl, R₂ is C1 alkyl and R₃ is C1 alkyl represents Licochalcone D.

That is, the present invention may provide a pharmaceutical composition for prevention or treatment of aging-related diseases, which includes Licochalcone D or a pharmaceutically acceptable salt thereof.

In Formula 1 above, compounds in which R₁, R₂ and R₃ are each independently C1 to C6 alkyl may have the same matrix and similar substituents, therefore, it is sufficiently predictable that these compounds may show similar functional effects because they may possess similar features in structural or chemical aspects. For example, it is considered that aging-related diseases preventing or treating effects of the compounds in which R₁, R₂ and R₃ are each independently C2 to C6 alkyl may be within a range sufficiently predictable from aging-related disease preventing or treating effects of the compounds in which R₁, R₂ and R₃ are each independently C1 alkyl.

In Formula 1 above, since the compounds in which R₁, R₂ and R₃ are each independently C1 to C6 alkyl may have the same matrix and short length alkyl substituents, it is sufficiently predictable that these compounds may show similar functional effects because they may possess similar features in structural or chemical aspects.

Licochalcone D is a component known to be contained in licorice, and may be a compound having a structure represented by Formula 2 below.

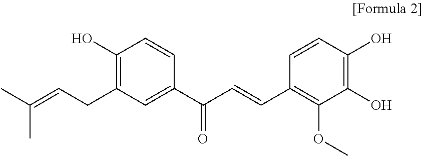 [SMILES: COc1c(/C=C/C(=O)c2ccc(O)c(CC=C(C)C)c2)ccc(O)c1O]

The composition of the present invention is effective in inducing autophagy through AMP-activated protein kinase (AMPK) activation, thereby exhibiting excellent effects of preventing or treating aging-related diseases.

Aging is a general concept including a deteriorated and changeable condition occurred due to a reduction in the structure and function of the body as getting older. Changes due to aging are highly diversified and may include, for example, a reduction in weight of different tissues and body weight due to a decrease in the number of actual cells, a change of connective tissues, a change in body composition, a reduction in elasticity of blood vessels or skin, functional weakness of organs, a deterioration in anti-disease or disease resilience including immune ability, a reduction of sensory functions, deteriorations in memory ability, learning ability and comparison ability, etc.

Aging-related diseases are not particularly limited as far as they are diseases occurred due to aging or diseases influenced by aging, and aging-related disease may be, for example, one selected from the group consisting of neurodegenerative diseases, cognitive functional disorder due to aging and skin-related diseases. The neurodegenerative disease may be, for example, one selected from the group consisting of dementia, Alzheimer's disease, Huntington's disease and Parkinson's disease. Cognitive function in regard to the cognitive functional disorder due to aging may be, for example, one selected from the group consisting of perception, memory, attention, speech comprehension, speech generation, reading comprehension, creation imagery, learning and reasoning. The skin-related disease may be, for example, one selected from the group consisting of skin wrinkles, photo aging, melanosis (sun tan), skin pigmentation, skin burn, skin inflammation and skin cancer.

The expression “pharmaceutically acceptable” means characteristics of not impairing physical properties as well as biological activity of a compound are exhibited without arousing significant stimulation in a subject, cell, tissue, etc. to which the compound or a composition including the compound is administered.

The expression “pharmaceutically acceptable salt” refers to a salt prepared using a specific compound according to the present invention, as well as acid or base relatively nontoxic thereto. The pharmaceutically acceptable salt may include, for example, acid addition salts or metal salts.

The acid addition salts may be formed from inorganic acids such as hydrochloric acid, nitric acid, phosphoric acid, sulfuric acid, hydrobromic acid, hydroiodic acid, nitrous or phosphorous acid, aliphatic mono and dicarboxylates, phenyl-substituted alkanoates, hydroxy alkanoates and alkane dioates, and non-toxic organic acids such as aromatic acids, aliphatic and aromatic sulfonic acids. These pharmaceutically non-toxic salts may include sulfate, pyrosulfate, bisulfate, sulfite, bisulfite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutyrate, caprate, heptanoate, propyolate, oxalate, malonate, succinate, suberate, sebacate, fumarate, maleate, butine-1,4-dioate, nucleic acid-1,6-dioate, benzoate, chlorobenzoate, methylbenzoate, dinitrobenzoate, hydroxybenzoate, methoxybenzoate, phthalate, terephthalate, benzene sulfonate, toluene sulfonate, chlorobenzene sulfonate, xylene sulfonate, phenylacetate, phenylpropionate, phenylbutyrate, citrate, lactate, β_hydroxybutyrate, glycolate, malate, tartrate, methane sulfonate, propane sulfonate, naphthalene-1-sulfonate, naphthalene-2-sulfonate or mandelate. For example, the acid addition salt may be obtained by dissolving the compound in an excess amount of aqueous acid solution and precipitating the salt using a hydrated organic solvent such as methanol, ethanol, acetone or acetonitrile.

The metal salt may be a sodium, potassium or calcium salt. The metal salt may be prepared using a base, for example, alkali-metal or alkaline earth metal salts may be obtained by dissolving the compound in an excess amount of alkali-metal hydroxide or alkaline earth metal hydroxide solution, filtering the non-dissolved compound salt, and evaporating and/or drying the filtrate.

The term “prevention” refers to a precautionary procedure that includes a slight, substantial or significant reduction in possibility of occurrence or recurrence of disease condition as well as overall prevention, and leads to a reduction in some extent of onset possibility of the disease condition to be prevented or the disease condition recurred or being recurred, wherein the extent of reduction in possibility means at least slight reduction.

The term “treatment” refers to a procedure that includes some extent of alleviation including slight alleviation, substantial alleviation or major relaxation as well as healing, and leads to beneficial effects on a subject or patient suffering from a disease condition to be treated, wherein the extent of relaxation means at least slight relaxation.

The pharmaceutical composition of the present invention may be formulated and used in the form of oral formulations such as powder, granules, tablets, capsules, suspension, emulsion, syrup, aerosol, etc., external applications, suppositories, and sterile injection, but it is not limited thereto.

Carriers, excipients and diluents able to be contained in the composition may include, for example, lactose, dextrose, sucrose, dextrin, maltodextrin, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate and mineral oil, but they are not limited thereto. Such formulations are produced using diluents or excipients such as fillers, extenders, binders, wetting agents, disintegrants, surfactants, etc., which are typically used in the art, but they are not limited thereto.

Solid formulations for oral administration may include tablets, pills, powder, granulates, capsules, etc., without limitation thereof, and such solid formulations may be prepared by admixing the compound as described above with at least one excipient, for example, starch, calcium carbonate, sucrose, lactose, gelatin and the like. Further, other than simple excipients, lubricants such as magnesium stearate, talc, etc. may also be used.

Liquid formulations for oral use may include suspending agents, oral liquids, emulsions, syrup and the like. Other than simple diluents commonly used in the art such as water and liquid paraffin, various excipients such as wetting agents, sweeteners, fragrances, preservatives, etc. may be used. Formulations for parenteral administration may include sterile aqueous solution, non-aqueous solvent, suspending agents, emulsions, freeze-dried preparations, suppositories and the like. The non-aqueous solvents or suspending agents used herein may include propylene glycol, polyethylene glycol, vegetable oil such as olive oil, injectable ester such as ethyl oleate, etc. As a base of the suppository, witepsol, macrogol, tween 60, cacao butter, laurin, glycerogelatin, and the like may be used.

The pharmaceutical composition of the present invention may be administered in a pharmaceutically effective amount. In the present invention, the expression “pharmaceutically effective amount” means an amount sufficient to treat a disease at a reasonable benefit/damage rate applicable to the medical treatment, and effective dose levels may be determined depending on types of disease of the patient, severity, activity of drug, sensitivity to drug, administration time, administration route and rate of release, duration of treatment, factors including concurrent medications, and other factors well known in the medical field. The pharmaceutical composition of the present invention may be administered as an individual therapeutic agent or in combination with other therapeutic agents, may be administered sequentially or simultaneously with conventional therapeutic agents, and may be administered in single or multiple doses. Taking all of the above factors into consideration, it is important to administer the pharmaceutical composition in an amount that can achieve maximum effects with a minimum amount without side effects, which may be easily determined by those skilled in the art.

With regard to the pharmaceutical composition of the present invention, the “effective amount” may vary depending on the age, gender, body weight of a patient. Generally, the composition may be administered in an amount of 1 to 6000 mg, and preferably 60 to 600 mg per 1 kg of body weight once or in three (3) divided doses. However, since the dose may be increased/decreased according to the administration route, severity of disease, gender, body weight, age, etc., the range of the present invention is not particularly limited in any manner by the above administration dose.

The present invention relates to an anti-aging food composition.

The food composition may include a compound represented by Formula 1 below or a food-acceptable salt thereof:

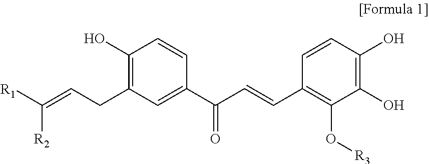

(wherein R₁ is H or C1 to C6 alkyl, R₂ is H or C1 to C6 alkyl, and R₃ is H or C1 to C6 alkyl).

The compound represented by Formula 1 above and the aging may be within the above-described range, but they are not limited thereto.

The composition of the present invention is effective in inducing autophagy through activation of AMP-activated protein kinase (AMPK), thereby attaining excellent anti-aging effects.

The anti-aging refers to any action to inhibit or delay aging symptoms described above, and specifically, refers to any action to reduce parameters relevant to the above-described aging at the least, for example, to reduce extents of the symptoms, wherein the action includes all actions or procedures to improve, alleviate or beneficially change aging symptoms by administering the composition of the present invention.

The expression “food-acceptable” means that characteristics of not impairing physical properties as well as biological activity of a compound are exhibited without arousing significant stimulation in a subject, cell, tissue, etc. to which the compound or a composition including the compound is administered.

The expression “food-acceptable salt” refers to a salt prepared using a specific compound according to the present invention, as well as acid or base relatively non-toxic thereto. The sitologically acceptable salt may include, for example, acid addition salts or metal salts wherein the acid addition salts and metal salts may be within the above-described range, but they are not limited thereto.

For the anti-aging purpose, the food composition may be prepared and processed in the form of tablets, capsules, powder, granules, liquid, pills, etc.

The food composition of the present invention may include any conventional food additive. Herein, suitability of the food composition as a food additive is judged on the basis of standards and criteria of corresponding items according to the General Regulations of the Food Additives and General Test Methods approved by the Food and Drug Administration, unless otherwise specified.

The items listed in the General Regulations of the Food Additives include, for example: chemical compounds such as ketones, glycine, calcium citrate, nicotinic acid and cinnamon acid; natural additives such as dark blue pigment, licorice extract, crystalline cellulose, high color pigment and guar gum; and mixed preparations such as sodium L-glutamate preparations, noodle-added alkaline chemicals, preservative preparations, and tar coloring preparations, and the like, but it is not limited thereto.

For example, the food composition in the form of tablets may be produced by mixing the composition with excipients, binders, disintegrants and other additives to prepare a mixture, granulating the mixture in any conventional manner, and then, compression molding the same along with addition of a lubricant or directly compression molding the mixture. Further, the food composition in the form of tablets may contain a flavor enhancer, or the like as necessary.

Among food compositions in the form of capsules, a hard capsule formulation may be produced by filling a typical hard capsule with a mixture of the composition and additives such as excipients, and a soft capsule formulation may be produced by filling a capsule base such as gelatin with a mixture of the composition and additives such as excipients. The soft capsule formulation may further contain a plasticizer such as glycerin or sorbitol, a colorant, a preservative, and the like as necessary.

A food composition in the form of pills may be produced by molding a mixture of the composition and excipients, binders, disintegrants, etc. according to any known method and, if necessary, may be enveloped with white sugar or other enveloping agents. Alternatively, the surface of the food may be coated with specific materials such as starch, talc and the like.

A food composition in the form of granules may be produced by granulating a mixture of the composition and excipients, binders, disintegrants, etc. according to a known method, and may contain a flavoring agent, a flavor enhancer, and the like as necessary.

The food composition may be beverages, meat, chocolate, foods, confectionery, pizza, ramen, other noodles, gums, candy, ice cream, alcoholic beverages, vitamin complexes and dietary supplements.

The food composition may be orally applied for use of nutritional supplements, and the application forms thereof are not particularly limited. For example, for oral administration, daily intake is preferably 5000 mg or less, and more preferably 2000 mg or less. Most preferably, the daily intake ranges from 5000 to 1500 mg or is 650 mg. When formulated into capsules or tablets, one capsule or tablet may be administered along with water once a day.

The present invention relates to an anti-aging cosmetic composition.

The cosmetic composition may include a compound represented by Formula 1 below or a cosmetically acceptable salt thereof:

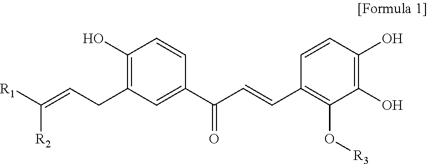

(wherein R₁ is H or C1 to C6 alkyl, R₂ is H or C1 to C6 alkyl, and R₃ is H or C1 to C6 alkyl).

The compound represented by Formula 1 above, aging and anti-aging may be within the above-described range, but they are not limited thereto.

The composition of the present invention is effective in inducing autophagy through activation of AMP-activated protein kinase (AMPK), thereby attaining excellent anti-aging effects.

The expression “cosmetically acceptable” means that characteristics of not impairing physical properties as well as biological activity of a compound are exhibited without arousing significant stimulation in a subject, cell, tissue, etc. to which the compound or a composition including the compound is administered.

The expression “cosmetically acceptable salt” refers to a salt prepared using a specific compound according to the present invention, as well as acid or base relatively non-toxic thereto. The cosmetically acceptable salt may include, for example, acid addition salts or metal salts wherein the acid addition salts and metal salts may be within the above-described range, but they are not limited thereto.

The anti-aging cosmetic composition of the present invention may further include components commonly used in the cosmetic composition other than active ingredients, and may include, for example, conventional supplements such as antioxidants, stabilizers, solubilizing agent, vitamin, pigments and aromatic essences, as well as carriers.

The cosmetic composition of the present invention may be prepared into any formulation generally produced in the art, and for example, may be formulated in the form of solution, suspension, emulsion, paste, gel, cream, lotion, powder, soap, surfactant-containing cleanser, oil, powder foundation, emulsion foundation, wax foundation, pack, massage cream, spray, etc., but it is not limited thereto. More specifically, the composition may be manufactured into any formulation such as softening beauty wash, nourishing beauty wash, nutritional cream, massage cream, essence, eye cream, cleansing cream, cleansing foam, cleansing water, pack, spray or powder.

When the formulation of the present invention is the paste, cream or gel, an animal oil, vegetable oil, wax, paraffin, starch, tragacanth, cellulose derivative, polyethylene glycol, silicone, bentonite, silica, talc, zinc oxide or the like may be used as a carrier component.

When the formulation of the present invention is the solution or emulsion, a solvent, solubilizing agent or emulsifying agent is used as the carrier component. For example, water, ethanol, isopropanol, ethyl carbonate, ethyl acetate, benzyl alcohol, benzyl benzoate, 1,3-butyl glycol oil, glycerol aliphatic ester, polyethylene glycol or sorbitan fatty acid ester may be used.

When the formulation of the present invention is the suspension, liquid diluents such as water, ethanol or propylene glycol, suspending agents such as ethoxylated isostearyl alcohol, polyoxyethylene sorbitol esters and polyoxyethylene sorbitan esters, microcrystalline cellulose, aluminum metahydroxide, bentonite, agar, tragacanth, or the like may be used as the carrier component.

When the formulation of the present invention is the powder or spray, lactose, talc, silica, aluminum hydroxide, calcium silicate or polyamide powder may be used as the carrier component. In particular, in the case of the spray, the composition may further include propellants such as chloro-fluorohydrocarbon, propane/butane or dimethyl ether.

When the formulation is the surfactant-containing cleanser, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamido betaine, aliphatic alcohol, fatty acid glyceride, fatty acid diethanolamide, vegetable oil, lanolin derivative, ethoxylated glycerol fatty acid ester, or the like may be used as the carrier component.

When the cosmetic composition of the present invention is the soap, surfactant-containing cleansing formulation or surfactant-free cleansing formulation, it may be applied to the skin and then wiped off, peeled off or washed with water. Specifically, the soap may be a liquid soap, powdered soap, solid soap or oil soap; the surfactant-containing cleansing formulation may be a cleansing foam, cleansing water, cleansing towel and cleanser pack and the surfactant-free cleansing formulation may be a cleansing cream, cleansing lotion, cleansing water and cleansing gel, but it is not limited thereto.

The present invention relates to a method for prevention or treatment of aging-related diseases.

The method for prevention or treatment of aging-related diseases may include: administering a compound represented by Formula 1 below or a pharmaceutically acceptable salt thereof to a subject in need thereof:

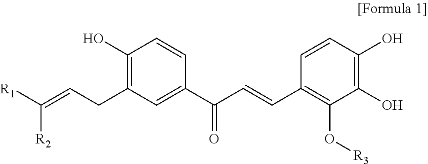

(wherein R₁ is H or C1 to C6 alkyl, R₂ is H or C1 to C6 alkyl, and R₃ is H or C1 to C6 alkyl).

The subject may include human and/or animals except for the human.

The subject may include subjects who have been diagnosed to have aging-related diseases or are at risk for the same, but it is not limited thereto.

The present invention relates to a stem cell protective composition.

The stem cell protective composition may include a compound represented by Formula 1 below or a physiologically acceptable salt thereof:

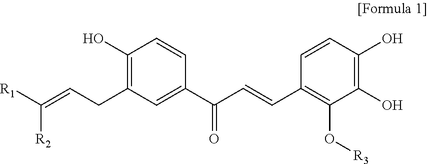

(wherein R₁ is H or C1 to C6 alkyl, R₂ is H or C1 to C6 alkyl, and R₃ is H or C1 to C6 alkyl).

The compound represented by Formula 1 above and aging may be within the above-described range, but they are not limited thereto.

The composition of the present invention has not only anti-oxidative activity but also autophagy-inducing effects through activation of AMP-activated protein kinase (AMPK), thereby attaining excellent stem cell protection effects.

The composition of the present invention may be used as a medium for stem cells.

The stem cells may include, for example, human bone marrow mesenchymal stem cells, but they are not limited thereto. In this regard, the mesenchymal stem cell is one of adult stem cells extracted from bone marrow and cord blood and known to exist in the number of about 1 million in the body. The mesenchymal stem cell has a shape of fibroblast (a spindle shape or a fusiform) and adhesive property, is possibly immortalized in vitro, and is characterized in that it may become differentiated matrix cell belonging to bone cell, cartilage cell, adipocyte, muscle cell and fibroblast linage, which are distinguishable from hematopoietic stem cells.

Stem cell protection means to protect the stem cell from oxidative stress or aging. Aging may be within the above-described range, but it is not limited thereto. Protecting cells from aging may be confirmed through a reduction in expression of aging-relevant factors of the cells, wherein the aging-relevant factors may include, for example, β-galactosidase, p16, p21, p53, etc. Further, protecting cells from aging may also be confirmed through activation of AMP-activated protein kinase (AMPK). Mechanistic target of rapamycin (mTOR) may be inhibited by AMPK activation. Further, when mTOR signal is inhibited, autophagy is activated, and thereby cell protection against aging can also be confirmed through autophagy activation. AMPK activation may be confirmed through an increase in a ratio of pAMPK expression to AMPK expression (pAMPK/AMPK), while autophagy activation may be confirmed through an increase in expression of LC3II (microtubule-associated protein 2 light chain 3) and/or BECN1 (beclin 1) or a decrease in expression of SQSTM1 (sequestosome 1).

The expression “physiologically acceptable” means that characteristics of not impairing physical properties as well as biological activity of a compound are exhibited without arousing significant stimulation in a subject, cell, tissue, etc. to which the compound or a composition including the compound is administered.

The expression “physiologically acceptable salt” refers to a salt prepared using a specific compound according to the present invention, as well as acid or base relatively non-toxic thereto. The physiologically acceptable salt may include, for example, acid addition salts or metal salts wherein the acid addition salts and metal salts may be within the above-described range, but they are not limited thereto.

Hereinafter, the present invention will be described in more detail by way of the following examples in order to concretely describe the present invention.

### Example

Materials and Methods

1. Chemicals and Reagents

Methylthiazolyldiphenyl-tetrazolium bromide (#M2128 MTT) assay; 2′,7′-dichlorofluorescin diacetate (#D6883), ascorbic acid (#A8960), H₂O₂ (#H1009), and D-Gal (#G0750) were purchased from Sigma-Aldrich (USA). S-β-gal(Senescence β-Galactosidase) staining assay kit (#9860) was obtained from Cell Signaling Technology (Danvers, Mass., USA). Lico D (#CFN99805; purity ≥98%) was purchased from ChemFaces (430056; Wuhan, Hubei) and Compound C was purchased from Calbiochem (Darmstadt, Germany). Radioimmunoprecipitation (RIPA) lysis buffer (Santa Cruz Biotechnology, Dallas, Tex., USA) and Pierce BCA protein assay kit and Hoechst 33342 Trihydrochloride Trihydrate (#H3570) were purchased from Thermo Fisher Scientific, USA, respectively. RNAiso Plus (#9109; Total RNA extraction reagent) and PrimescriptTM II 1st strand cDNA synthesis kits (#6210A) were purchased from Takara Bio Inc. (Japan). Primary antibodies including p16 (#sc-1661), p21 (#sc-397), p53 (#sc-6243), Caspase-3 (#sc-7148); MAP LC3α/β (#sc-398822); BECN1 (#sc-48341); SQSTM1 (#sc-48402) and GAPDH (#sc-365062) were acquired from Santa Cruz Biotechnology, INC. (TX, USA). The anti-caspase 3 active form (#AB3623; 1:500) was purchased from Merck Millipore, Germany and AMPKα (#5832) and p-AMPK (#2535) were purchased from Cell Signaling Technology (USA). The appropriate HRP-conjugated secondary antibodies, mouse anti-rabbit (#sc-2357), and mouse anti-goat (#sc-2354) antibodies were purchased from Santa Cruz Biotechnology, Inc. (Dallas, Tex., USA) and the horse anti-mouse (#7076) antibody was from Cell Signaling Technology (USA). ECL Western blotting detection reagents (RPN2209) were purchased from GE Healthcare (Buckinghamshire, UK).

2. hBM-MSCs Cell Culture

Human bone marrow derived mesenchymal stem cells (hBM-MSCs) were acquired from Cell Engineering for Origin (CEFO), Seoul, Korea. The cells were free from bacterial, viral, and mycoplasmal contamination. The cells were characterized by flow cytometry analysis, which revealed the CD73⁺, CD105⁺, and CD31⁻ phenotype (data not shown). The cells were grown in Dulbecco's modified Eagle medium (DMEM) (Gibco, Life Technologies, Grand Island, N.Y., USA) supplemented with 10% FBS (Gibco, Life Technologies, USA), L-glutamine, and 1% penicillin/streptomycin solution (Lonza, Walkersville, Md., USA). The cells were manipulated under sterile conditions in a humidified incubator at 37° C. with 5% CO₂ and 95% air. The cells were subcultured as soon as they reached confluence. The cells were serially monitored under bright field microscopy (Nikon Eclipse TS100; Tokyo, Japan), and the media was changed every 3 d.

3. Cell Viability Assay

The cytotoxic and protective effects of Lico D in hBM-MSCs were determined using the methylthiazolyldiphenyl-tetrazolium bromide (MTT) test, according to the manufacturer's instructions. To check the cytotoxic effect, hBM-MSCs were grown in a 96-well plate at a density of 8×10³ cells per well. The next day, the cells were treated with different concentrations of Lico D (1-8 μg/mL) for 12 h, followed by incubation with MTT solution for 2 h, and the formazan crystals were dissolved using dimethyl sulfoxide (DMSO). Cell viability was assessed using a spectrophotometer (Multiskan FC, Thermo Fisher Scientific). For the protective effect of Lico D, cells were pre-treated with 1 μg/mL of Lico D for 12 h followed by incubation with different concentrations of H₂O₂ (0.5-1 mM) for 1 h. Following H₂O₂ treatment, cells were treated with MTT solution for 2 h, and cell viability was evaluated.

4. Detection of Intracellular ROS (Reactive Oxygen Species)

To quantify the production of intracellular ROS, we used cell permeable substrate 2′,7′-dichlorofluorescin diacetate (DCFH-DA), which can be converted to highly detectable fluorescent 2′,7′-dichlorofluorescein upon oxidation. The cells were cultured in 12-well plates and 96-well plates for 24 h. After 24 h of culture, the cells were treated with 1 μg/mL of Lico D for 12 h and incubated with 20 μM DCFH-DA for 30 min. The cells were then washed with a phosphate-buffered saline (PBS) solution and incubated with H₂O₂ (0.7 mM) for 1 h at 37° C. The cells were then fixed with 4% paraformaldehyde at room temperature (RT) for 15 min and the nuclei were stained with Hoechst. Then, the cells were observed under a fluorescent microscope (Nikon Eclipse Ti2; Japan), and the images were captured (Nikon DS-Ri2; Japan) and analyzed using the Image J software. To analyze the apoptotic markers, the cells were pretreated with 1 μg/mL of Lico D followed by incubation with H₂O₂ (0.7 mM) for 1 h at 37° C., and then the proteins were isolated as described below.

5. Oxidative Stress-Induced Senescence

H₂O₂ was used to induce oxidative stress-induced cell cycle arrest and senescence. Briefly, the cells were exposed to 200 μM H₂O₂ for 2 h and cultured for 2 d without H₂O₂. In the second treatment with H₂O₂, the cells were split 1:3, exposed to 200 μM H₂O₂ for 2 h, and cultured with either normal media (control), Lico D (1 μg/mL), ascorbic acid (500 μM AA; a positive control), or the compound C (0.5 μM; AMPK inhibitor) for 72 h. Cellular senescence was confirmed by SA-β-gal assay and Western blotting as described below.

6. Senescence-Associated β-Galactosidase (SA-β-Gal) Staining

Senescence-associated β-galactosidase positive cells were identified using an SA-β-gal assay kit (Cell Signaling Technology, Danvers, Mass., USA) according to the manufacturer's instructions. Briefly, after 72 h of treatment, cells were washed with phosphate-buffered saline (PBS) and fixed with 1× fixative for 10-15 min at room temperature. The β-galactosidase staining solution (pH 6.0) was added to the plates and incubated overnight in a dry incubator at 37° C. without CO₂. The next day, the β-Gal positive cells were observed under a light microscope (Nikon Eclipse TS100; Tokyo, Japan) and captured using a Canon i-Solution IMTcam3 digital camera (Tokyo, Japan).

7. Immunoblotting Analysis

Total proteins were extracted with RIPA (radioimmunoprecipitation) lysis buffer system containing phenylmethylsulfonyl fluoride (PMSF), sodium orthovanadate (Na₃VO₄), and protease inhibitor cocktail (Santa Cruz Biotechnology) at 4° C. for 30 min, and the samples were centrifuged (spun down) at 16,000×g for 20 min. The total protein concentration was quantified using the Pierce BCA Protein Assay Kit (Thermo Fisher Scientific). Proteins (30-50 μg) were loaded and separated via sodium dodecyl sulphate-polyacrylamide gel electrophoresis (SDS-PAGE) gel electrophoresis, followed by blotting on a PVDF membrane (GE Healthcare, Germany). The membranes were blocked with 1% blocking solution containing non-fat dry milk in TBST for 1 h 30 min at RT to prevent non-specific binding of primary antibodies. Subsequently, the membranes were incubated overnight at 4° C. with the corresponding primary antibodies. Secondary antibodies were conjugated with horseradish peroxidase and visualized using an enhanced chemiluminescence detection kit (GE Healthcare). Densitometry analysis was performed using the ImageJ software.

8. Animals and Administration of Drugs

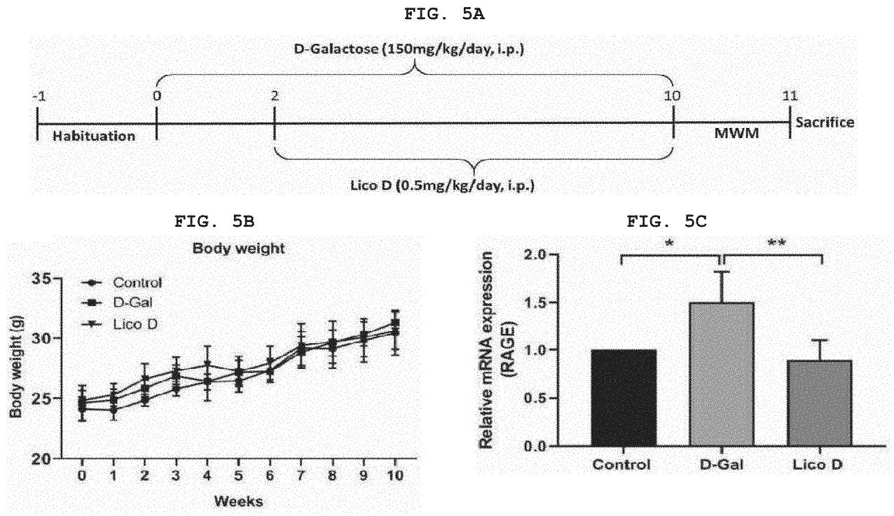

Six-week-old male C57BL/6 mice (weighing 22±2 g) were purchased from Samtako Bio Korea Co., Ltd. (Osan, Gyeonggi, Korea) and maintained at 23-25° C. under a 12-h light and 12-h dark cycle with free access to food and water in a pathogen-free facility. All animal experiments were approved by the Institutional Animal Care and Use Committee of Chosun University (CIACUC2020-S0009). After a week of acclimation, the animals were randomly divided into three groups (four mice per group): normal control group (PBS alone), D-Galactose model group (150 mg/kg/day), and D-Galactose +Lico D (0.5 mg/kg/day). The mice were intraperitoneally injected with either PBS or D-Gal for 10 weeks and from the third week onwards, the mice were intraperitoneally injected with Lico D for 8 weeks (FIG. 5A). Body weight was measured every week until the end of the experiment. Thereafter, the MWM (Morris water maze) test was performed for one week.

9. RNA Extraction and Quantitative Reverse Transcription-Polymerase Chain Reaction (qRTPCR)

RNAisoPlus (Takara) was used to isolate total RNA from the hippocampus and heart tissues. Total RNA (2.5 μg) was then reverse-transcribed using PrimescriptTM II 1st strand cDNA synthesis kit (Takara) and quantified using the Power SYBR Green PCR Master mix (Applied BioSystems). To amplify β-actin and RAGE, the mouse primers in Table 1 below were used.

TABLE 1

| | | | Sequence |
|---|---|---|---|
| Gene | Sequence | | number |
| β-actin | Forward | 5′-CCACCATGTACCCAGGCA | 1 |
| | | TT-3′ | |
| | Reverse | 5′-CGGACTCATCGTACTCCT | 2 |
| | | GC-3′ | |
| RAGE | Forward | 5′-AGGTGGGGACATGTGTGT | 3 |
| | | C-3′ | |
| | Reverse | 5′-TCTCAGGGTGTCTCCTGG | 4 |
| | | TC-3′ | |

Real-time PCR reactions were performed using the StepOne™ Real-Time PCR system (Applied Bio Systems) and the primer pairs were synthesized by GenoTech (Daejeon, Korea) or IDT (Integrated DNA Technologies, Coralville, Iowa, USA).

10. Statistical Analyses

All data are presented as the mean±standard deviation from at least three or more biological replicates. The differences between control and treatment groups were assessed by Student's t-test, several groups were used in One-way ANOVA using Turkey's test for post-hoc comparison analysis. A p-value of <0.05 was considered statistically significant, and a p-value of <0.01 was considered very significant.

Results

1. Lico D Protects the hBM-MSCs Against Oxidative Stress

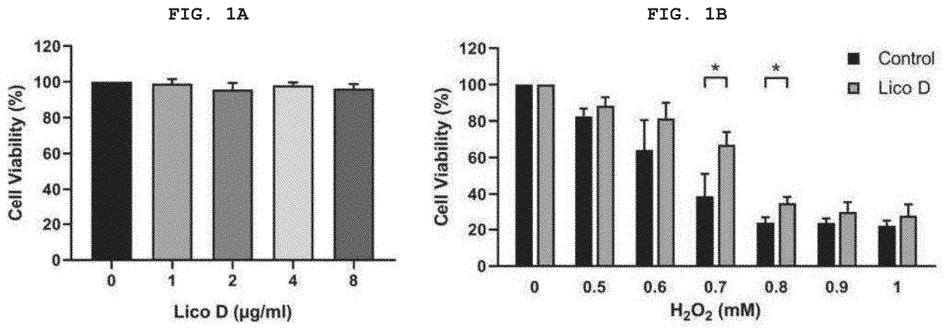

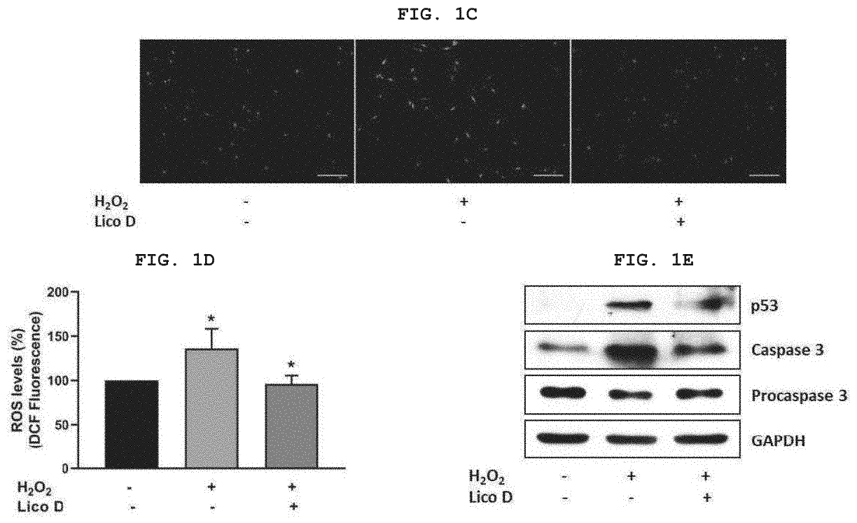

Initially, in order to determine the effect of Lico D on hBM-MSC viability, we treated hBM-MSCs with different concentrations of Lico D (0-8 μg/mL) for 12h and cell viability was measured. As shown in FIG. 1A, there were no significant changes in cell viability upon Lico D treatment. However, it has been previously reported that Lico D has anticancer activity in a dose-dependent manner against human melanoma, oral squamous cell carcinoma and lung adenocarcinoma. Therefore, we chose the lowest concentration of Lico D (1 μg/mL) for our further in vitro experiments. It is well known that increased oxidative stress is one of the main causes of senescence of stem cells by decreasing self-renewal and differentiation and increasing apoptosis and stem cell depletion. Hydrogen peroxide (H₂O₂) was used in vitro to induce oxidative stress. The antioxidant properties of Lico D were evaluated against H₂O₂ in hBM-MSCs. Lico D-pretreated hBM-MSCs were exposed to different concentrations of H₂O₂ (0-1 mM) and cell viability was measured. The results indicated that cell viability was significantly increased in Lico D-pretreated hBMMSCs (FIG. 1B). Increased endogenous ROS levels were observed in long-term culture of hBM-MSCs and induced apoptosis in human adipose derived mesenchymal stem cells (hAD-MSCs). Therefore, we examined the effect of Lico D on ROS generation and cell death. As expected, intracellular ROS levels were significantly increased upon H₂O₂ treatment, whereas they were significantly reduced in Lico D-pretreated hBM-MSCs (FIGS. 1C and 1D). Furthermore, these protective effects of Lico D were also confirmed by the expression levels of apoptotic proteins, such as p53 and cleaved caspase 3 (FIG. 1E). Both markers were increased upon treatment with H₂O₂, whereas the expression of these markers was reduced in Lico D-pretreated hBM-MSCs. These results indicate that Lico D has an anti-apoptotic effect on oxidative stress. Collectively, these results suggest that Lico D protects hBMMSCs against oxidative stress by inhibiting excess intracellular ROS production and cell death.

2. Lico D Reduces the Oxidative Stress Induced Senescence in hBM-MSCs

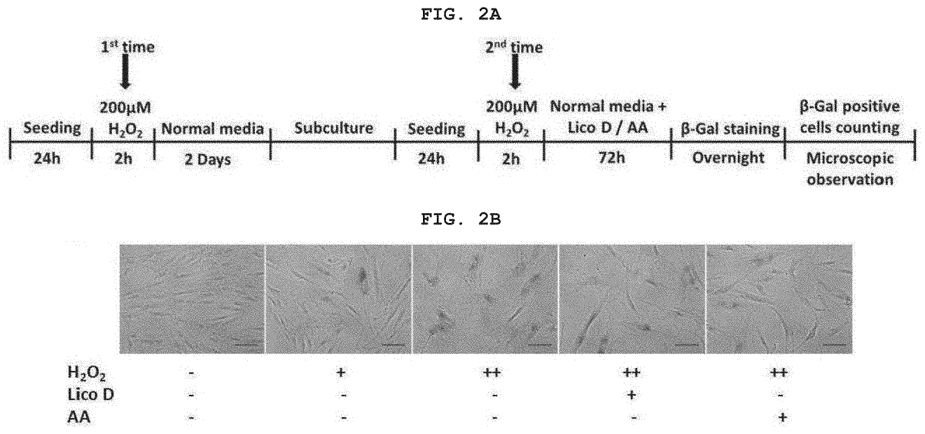

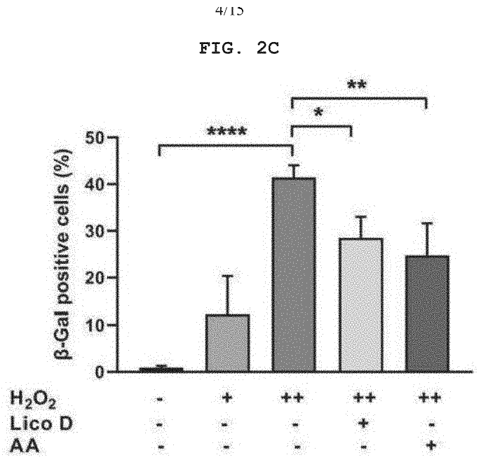

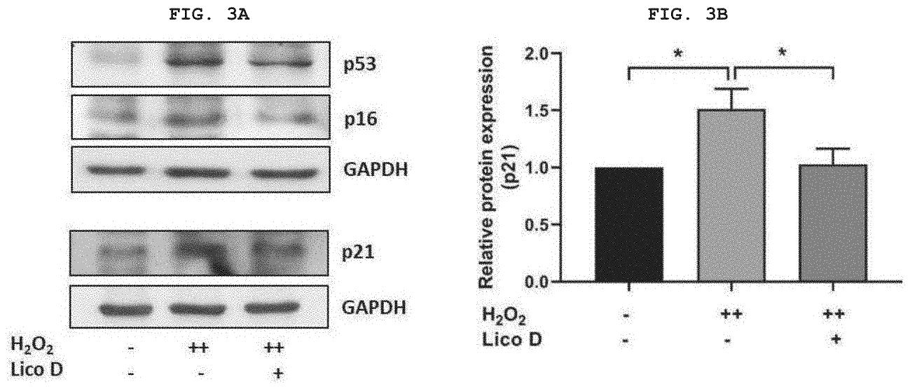

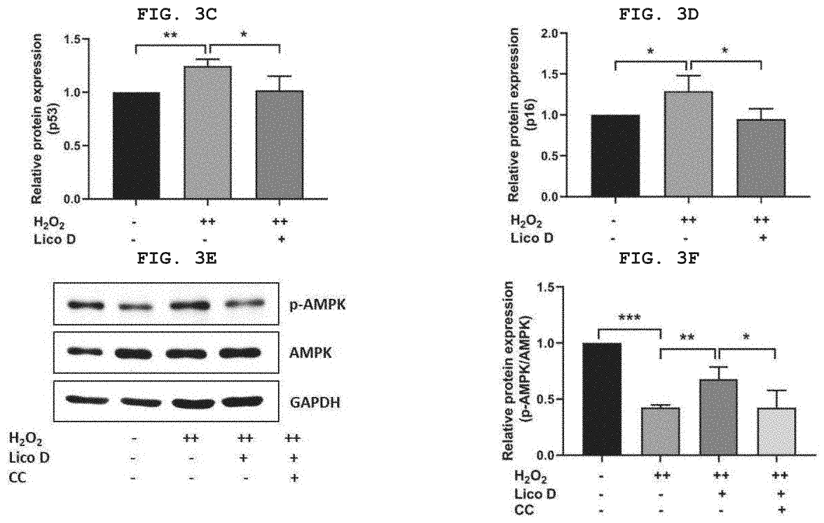

Excessive production of intracellular ROS may contribute to the progression of replicative senescence in stem cells and the accumulation of apoptotic insensitive cells. To investigate the protective effect of Lico D in hBM-MSC senescence, the cells were subjected to stress-induced premature senescence (SIPS), particularly the oxidative stress-induced senescence model. As shown in FIG. 2B, the oxidative stress-induced senescence cells became enlarged, flattened, and highly SA-β-gal-positive cells. The accumulation of SA-β-gal positive cells was significantly augmented in the double time H₂O₂ treated hBM-MSCs (40%) when compared with single time treated hBMMSCs (12%) (FIG. 2C). It is because a small fraction of cells can recover from oxidative stress and re-enter the cell cycle with a single H₂O₂ treatment. Therefore, we chose a double-time H₂O₂ treatment for further studies. As expected, the number of SA-β-gal-positive cells was significantly reduced by treatment with Lico D (13%) and ascorbic acid (17%, a positive control). Furthermore, the anti-senescence effects of Lico D were evaluated by the expression of well-known senescence markers, including p53, p16, and p21. As shown in FIGS. 3A-3D, the expression of senescence markers was increased in double time H₂O₂ treated hBM-MSCs, while it was reduced in Lico D-treated hBM-MSCs. These results suggest that Lico D not only protects hBM-MCSs from oxidative stress, but also reduces oxidative stress-induced senescence in hBM-MSCs.

3. Lico D Reduces the Oxidative Stress Induced Senescence Via Activation of AMPK and Autophagy

Inactivation of AMPK is known to cause senescence by oxidative stress, whereas pharmacological activation of AMPK prevents the occurrence of senescence due to oxidative stress. With this in mind, we assessed the role of AMPK in our senescence model. The results showed that AMPK activation was significantly decreased in the oxidative stress-induced senescence model (FIGS. 3E and 3F), while AMPK activation was significantly restored in Lico D-treated senescent cells. Furthermore, this effect was confirmed using compound C (CC), a potential AMPK inhibitor. As expected, Lico D-mediated AMPK activation was abolished by treatment with CC (FIGS. 3E and 3F). These data suggest that AMPK activation by Lico D can reduce oxidative stress-induced senescence in hBM-MSCs, and this reduction could be prevented by CC.

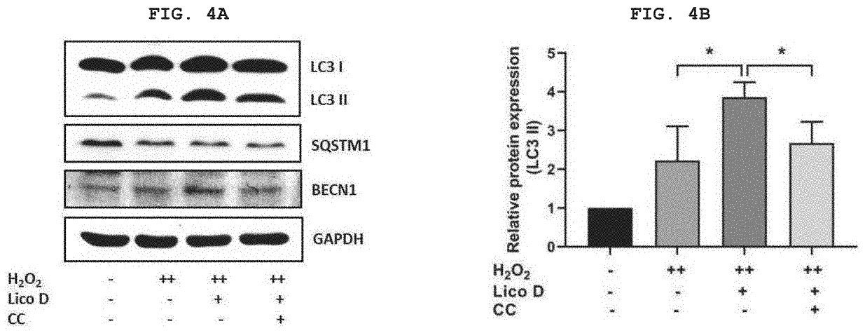

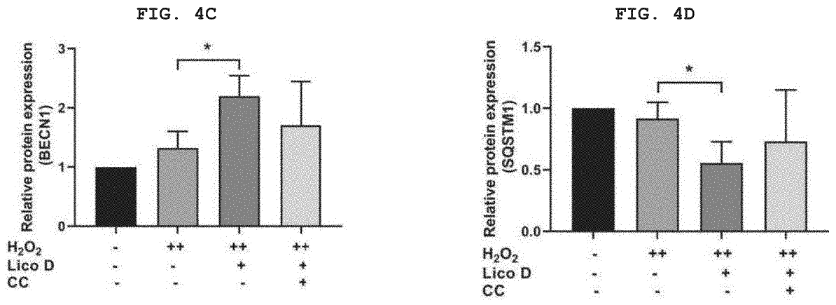

Increased oxidative stress and loss of autophagy are one of the major causes of cellular senescence and age-related diseases. According to previous studies, activation of AMPK is sufficient to restore autophagy flux in oxidative stress-induced senescent cells. Further, we extended our study to explore the role of Lico D in autophagy activation. The results showed that Lico D treatment significantly increased the expression levels of LC3 and BECN1 and decreased the expression of SQSTM1 (FIGS. 4A-4D). To verify the involvement of AMPK in autophagy, we used the CC. Interestingly, we found that AMPK-mediated autophagy was inhibited by treatment with CC. Taken together, our findings indicate that Lico D activates autophagy through AMPK and reduces oxidative stress-induced senescence.

4. Effect of Lico D on Body Weight in D-Gal Induced Aging Mice

The animal's body weight was measured every week until the experiment ended. No significant differences were observed in either control or D-Gal or Lico D-injected animals (FIG. 5B).

5. Lico D Reduces RAGE (Receptor for Advanced Glycation Endproducts) Expression in D-Gal Induced Aging Mice

Advanced Glycation Endproducts (AGEs) are a heterogeneous composite compound group formed by non-enzymatic reaction of reduced sugar associated with protein, lipid and amino groups of nucleic acid. It is known that AGE bound to a receptor of the above compound group, RAGE, increases oxidative stress and causes neuro-inflammation, neuro-degeneration and amnestic disorder. To identify the role of Lico D in hippocampal inflammation, we screened the aging-related inflammation marker RAGE. Our data showed that D-Gal treatment increased the expression of RAGE at the mRNA level compared to that in the control group (FIG. 5C). Treatment with Lico D significantly reduced RAGE expression compared to that in D-Galtreated mice. Our results suggest that Lico D can reduce D-Gal-induced hippocampal inflammation by reducing RAGE expression. In summary, when Lico D is administered, hippocampal inflammation is reduced thus to improve D-Gal induced spatial learning and memory deficits.

6. Spatial Learning and Memory Improving Effects of Licochalcone D in D-Gal Induced Aging Mice

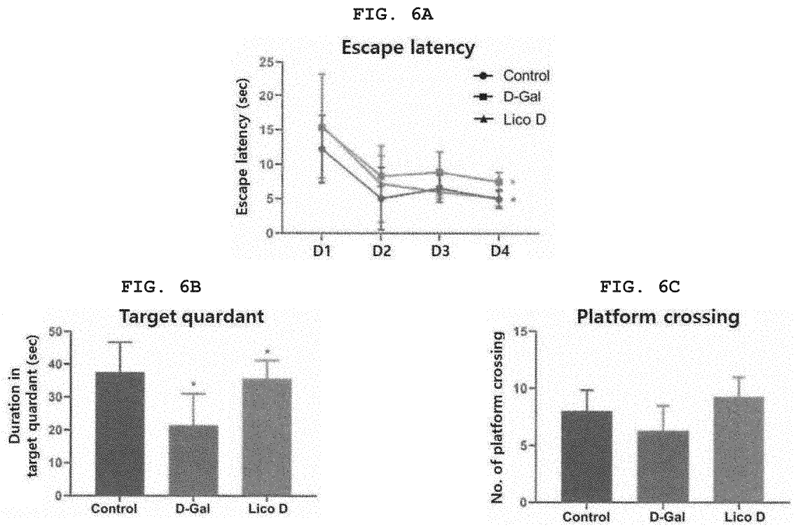

Escape latency, duration in target quadrant and the number of times of crossing target platform were investigated through a Morris water maze (MWM) test. As a result of measuring the escape latency of each mouse everyday (FIG. 6A), the Lico D treated mouse showed the escape latency over time, which is similar to that of the control (normal mice). This corresponded to a markedly short time, as compared to treatment of D-Gal treated mouse (FIG. 6A). With regard to the duration in target quadrant, it was also confirmed that the Lico D treated mouse showed to be similar to the control, and stayed in the target quadrant for a significantly longer time than the D-Gal treated mouse (FIG. 6B). Further, it was confirmed that Lico D treated mouse had a tendency of increasing the number of times of the target platform crossing, as compared to D-Gal treated mouse (FIG. 6C).

7. AMPK Activation by Lico D Ameliorates Heart and Hippocampus Senescence in D-Gal Induced Aging Mice

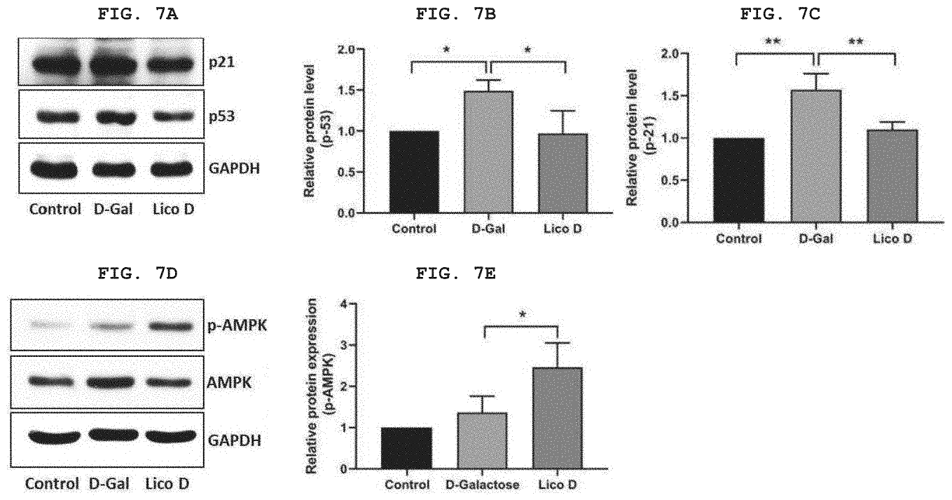

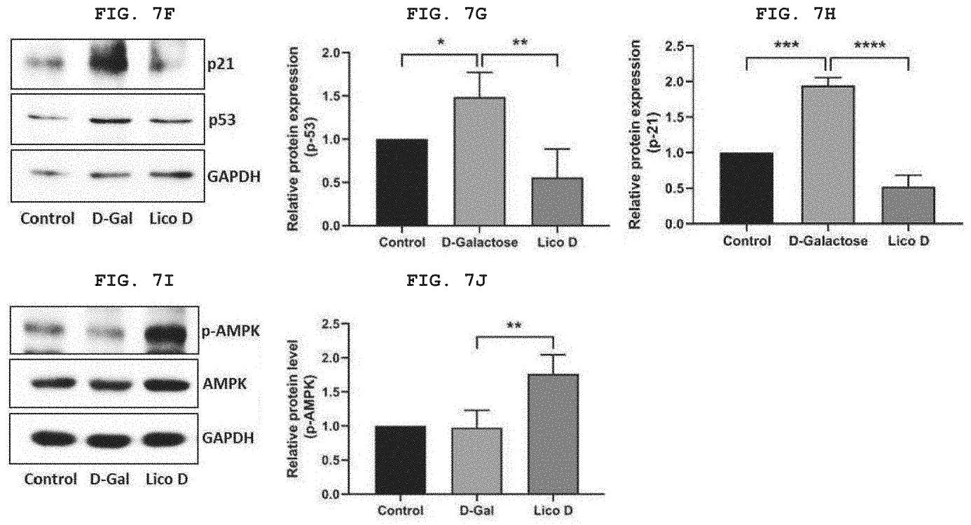

Oxidative stress can activate cellular senescence through various signaling cascades, but ultimately activate p53, p16, or both, and mediate cell cycle arrest. Aging mediated by the activation of the tumor suppressor p53 and its target p21 was significantly increased in response to D-Gal-induced oxidative stress in the heart and hippocampal tissues of animal models. With this in mind, we first examined the expression of senescence markers p53 and p21 in the heart and hippocampus tissues. As expected, the expression levels of p53 and p21 were unregulated in D-Gal-injected mice compared to those in the vehicle (FIGS. 7A-7C heart, and FIGS. 7F-7H hippocampus). More importantly, their expression was significantly reduced in the Lico Dtreated group. These data suggest that Lico D ameliorates heart and hippocampal senescence in D-Gal-induced aging mice. The longevity pathway mediated by AMPK may reduce the burden of senescence in the heart and hippocampus. Therefore, we analyzed the effect of Lico D on AMPK activation in the heart and hippocampus. There were no significant changes in the AMPK levels in the heart and hippocampus tissues of D-Gal-treated mice (FIGS. 7D and 7E hearts; and FIGS. 71 and 7J hippocampus). However, when compared to the D-Gal treatment, AMPK activation was significantly increased in both tissues of Lico D. These results suggest that Lico D reduces senescence by activating AMPK in D-Gal-induced aging mice.

8. Lico D Activates Autophagy in D-Gal Induced Aging Mice Heart Tissue

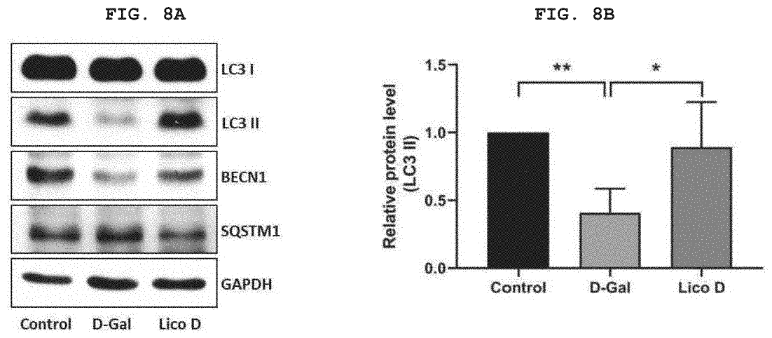

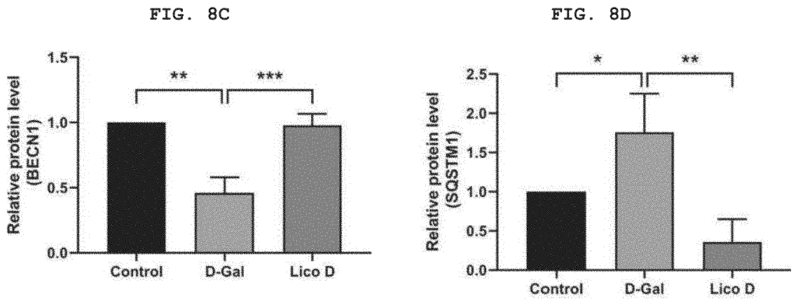

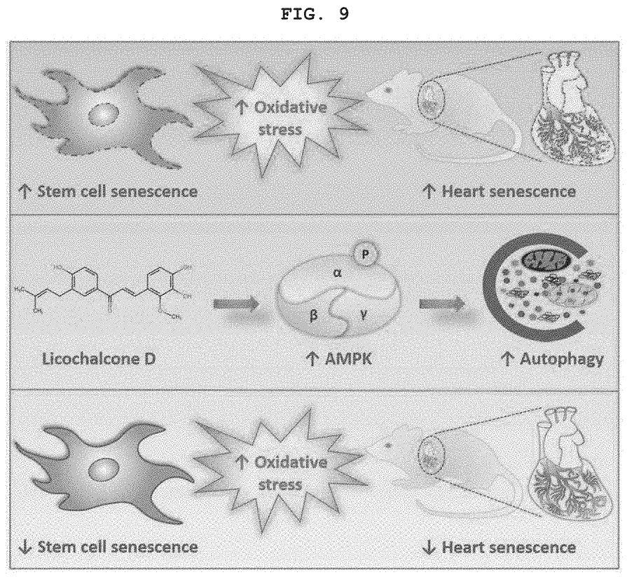

To confirm the effect of Lico D on autophagy activation, we analyzed the expression levels of autophagy markers in our mouse model. This is because autophagy disorders can accelerate the aging process. To facilitate the assessment of autophagy in the heart and hippocampus, the expression levels of LC3 II, BECN1 and SQSTM1 were identified. As shown in the FIGS. 8A-8D autophagy activation was significantly decreased in D-Gal-treated mice heart tissue compared to that in the control group. Lico D administration markedly increased the expression of these autophagy markers in heart tissue. Collectively, these results reinforce that Lico D reduces heart senescence by activating AMPK and autophagy in a D-Gal-induced aging mouse model.

### REFERENCE TO AN ELECTRONIC SEQUENCE LISTING

A sequence listing electronically submitted on Dec. 30, 2022 as a XML file named 20221230_Q89922LC44_TU_SEQ.XML, created on Oct. 13, 2022 and having a size of 6,171 bytes, is incorporated herein by reference in its entirety.

1-6. (canceled)

7. A method for treatment of aging-related disease, the method comprising administering a pharmaceutical composition comprising a compound represented by Formula 1 or a pharmaceutically acceptable salt thereof to a subject in need thereof: 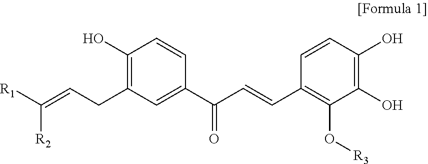
wherein R₁ is H or C1 to C6 alkyl, R₂ is H or C1 to C6 alkyl, and R₃ is H or C1 to C6 alkyl.

8. The method of claim 7, wherein the aging-related disease is at least one selected from the group consisting of neurodegenerative disease, cognitive functional disorder due to aging, and skin-related disease.

9. The method of claim 7, wherein the aging-related disease is the neurodegenerative disease selected from the group consisting of dementia, Alzheimer's disease, Huntington's disease and Parkinson's disease.

10. The method of claim 7, wherein the aging-related disease is the cognitive functional disorder which is a disorder related to at least one selected from the group consisting of perception, memory, attention, speech comprehension, speech generation, reading comprehension, creation imagery, learning and reasoning.

11. A method for anti-aging, the method comprising administering a food composition comprising a compound represented by Formula 1 or a sitologically acceptable salt thereof to a subject in need thereof: 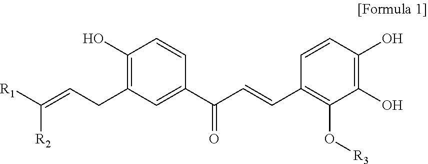
wherein R₁ is H or C1 to C6 alkyl, R₂ is H or C1 to C6 alkyl, and R₃ is H or C1 to C6 alkyl.

12. A method for anti-aging, the method comprising applying a cosmetic composition comprising a compound represented by Formula 1 or a cosmetically acceptable salt thereof to a subject in need thereof: 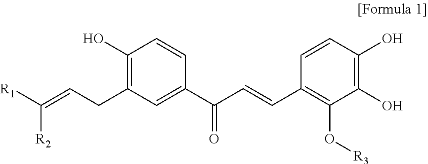
wherein R₁ is H or C1 to C6 alkyl, R₂ is H or C1 to C6 alkyl, and R₃ is H or C1 to C6 alkyl.
